(19) United States
(12) Patent Application Publication
(10) Pub. No.: US 2005/0155097 A1
(43) Pub. Date: Jul. 14, 2005

(54) Eosinophil-deficient transgenic animals

(76) Inventors: James J. Lee, Scottsdale, AZ (US); Nancy A. Lee, Scottsdale, AZ (US)

Correspondence Address: FISH & RICHARDSON PC, 225 FRANKLIN ST, BOSTON, MA, 02110, US

(21) Appl. No.: 10/755,599

(22) Filed: Jan. 12, 2004

(51) Int. Cl. A01K 67/027

(52) U.S. Cl. 800/18

(57) ABSTRACT

The technologies described herein are based on the discovery that expression of a toxin gene under control of an eosinophil-specific promoter can cause the ablation of eosinophils in a transgenic animal. Accordingly, the nucleic acid constructs featured in the invention are used to generate eosinophil-deficient transgenic animals that are useful for the study of pathologies and treatments relating to tissues and organ systems that typically contain eosinophils.

### GOVERNMENT SUPPORT

The work described herein was carried out, at least in part, using funds from the U.S. government under grant number HL-65228 awarded by the National Institutes of Health. The government may therefore have certain rights in the invention.

### TECHNICAL FIELD

The invention relates to transgenic non-human mammals that express a nucleic acid sequence containing a toxin gene under control of an eosinophil-specific promoter such that the transgenic animal lacks eosinophils.

### BACKGROUND

The management of asthma has changed significantly over the past decade reflecting the recognition of coincident chronic pulmonary inflammation. The wide variability in etiology and presentation of symptoms is anchored by three common characteristics: reversible variable airflow limitations, specific airway histopathologies, and airway hyperresponsiveness (AHR) (i.e., the development of bronchoconstriction in response to nonspecific inflammatory stimuli) (Bochner et al., Ann. Rev. Immun. 12:295, 1994). The onset and progression of allergic asthma is accompanied by overlapping, and often concurrent, inflammatory responses orchestrated by CD4⁺ T_{H}2 lymphocytes, including T cell mediated help of antigen-specific immunoglobulin production, expression of TH2 proinflammatory cytokines, the release of chemokines, and increases in adhesion molecule receptors on activated vascular endothelial cells. These T cell dependent pulmonary changes are also characterized by cellular infiltrates and the subsequent histopathologies believed to be the underlying cause(s) of the accompanying airway obstruction and lung dysfunction. In particular, the differential recruitment of eosinophils to the airway mucosa and lumen are common features of allergic respiratory disease, occurring in >75% of reported cases (Tomassini et al., J. Allergy Clin. Immunol. 88:365, 1991). This selective recruitment suggests that pulmonary pathologies arise, in part, as a consequence of eosinophil effector functions (EEFs). Indeed, studies have implicated eosinophils as immunoregulative cells modulating the inflammatory response as well as proinflammatory cells whose activities lead to epithelial desquamation, airway smooth muscle perturbation, and tissue remodeling (see for example, Underwood et al., Eur. Resp. Jour. 8:2104, 1995)). The use of mouse models has allowed for the dissection of immune pathways of allergic inflammation, including the definition of causative cell types and the identification of cytokine/chemokine ligands as well as their receptors. Moreover, the ability of these models to develop allergen-induced histopathologies and lung dysfunction has led to the widespread use of mice as models of human allergic inflammation.

### SUMMARY

The technologies described herein are based on the discovery that expression of a toxin gene under control of an eosinophil-specific promoter can cause the ablation of eosinophils in a transgenic animal. Accordingly, the nucleic acid constructs featured in the invention are used to generate eosinophil-deficient animal models that are useful for the study of pathologies and treatments relating to tissues and organ systems that typically contain eosinophils.

One feature of the invention is a transgenic non-human mammal, such as a rodent (e.g., a mouse or a rat), that is substantially free of eosinophils and otherwise retains a normal set of blood cells. For example, the transgenic non-human mammal can have a substantially normal level of red blood cells (RBCs). A “substantially normal level of RBCs” is the number of RBCs in a healthy mouse (e.g., about 10-13 RBC/mm³ of blood (×10⁻⁶). The transgenic non-human mammal contains a nucleic acid construct that includes a first nucleic acid sequence operably linked to a second nucleic acid sequence that is heterologous to the first nucleic acid sequence. The first nucleic acid sequence can promote eosinophil-specific expression of the second nucleic acid sequence, and the second nucleic acid sequence can encode a cell toxin. The first nucleic acid sequence can be an eosinophil peroxidase (EPO) promoter, such as SEQ ID NO:3 (FIG. 3), or a fragment thereof (Horton et al., J. Leukoc. Biol. 60:285-294, 1996). The cell toxin encoded by the second nucleic acid sequence can be a diphtheria toxin A chain having, for example, the amino acid sequence of SEQ ID NO:2 (FIG. 2). In other alternatives, the cell toxin can be Pseudomonas exotoxin A, ricin, or α-sarcin.

As used herein, a non-human mammal that is “substantially free of eosinophils” is a mammal that typically displays no histologically detectable eosinophils.

Other features of the invention include methods for investigating a role(s) for eosinophils in pulmonary physiology. The methods include: (i) providing a transgenic non-human mammal, such as one described above; (ii) exposing the transgenic non-human mammal to a pulmonary effector; (iii) comparing lung tissue from the exposed transgenic non-human mammal to lung tissue from a control non-human mammal; and (iv) investigating the role of eosinophils in pulmonary physiology. As used herein, a “pulmonary effector” is any agent that causes a physiological change in the lungs. For example, a pulmonary effector can be an allergen that induces pulmonary allergic disease. The control non-human mammal can be, for example, a non-transgenic non-human mammal exposed to the pulmonary effector; a non-transgenic non-human mammal not exposed to the pulmonary effector; or a transgenic non-human mammal not exposed to the pulmonary effector. Similar methods can be used to investigate a role(s) for eosinophils in the physiology of other tissues where eosinophils are localized, such as in the uterus, the thymus, and the gut (e.g., intestines, such as the small intestines). The methods can include exposing the tissue(s) to test compounds that may elicit a differential response in eosinophil-deficient and wildtype mammals. As used herein, “physiology” refers to the function of a tissue or organ, including the mechanical, physical, and biochemical functions, and any pathologies thereof.

Screening assays are also features of the invention. For example, a method of classifying a test compound as a positive or negative drug candidate is provided. According to one exemplary method, a transgenic non-human mammal, such as a transgenic non-human mammal described herein, is contacted with a test compound. An organ or tissue of the transgenic non-human mammal is then tested for a presence, absence, or degree of physiological change. Based on the detected presence, absence, or degree of physiological change, the test compound can be classified as a positive or negative drug candidate. The organ or tissue tested in these methods typically contains eosinophils. For example, the organ or tissue can be one or more of the group consisting of lung, gut (e.g., intestines), thymus, or uterine tissue.

Other features of the invention include nucleic acid constructs containing a first nucleic acid sequence operably linked to a heterologous second nucleic acid sequence. The first nucleic acid sequence can promote eosinophil-specific expression of the second nucleic acid sequence, and the second nucleic-acid sequence is operably linked to a nucleic acid sequence containing at least one intron. For example, at least a fragment of a human growth hormone gene (FIG. 1) including at least one intron can be fused to the 3′ end of the second nucleic acid. A fragment of the human growth hormone gene fused to the second nucleic acid can contain multiple exons and intervening introns. A nucleic acid construct featured in this invention can also contain a polyadenylation signal. The first nucleic acid sequence can have the sequence of the eosinophil peroxidase (EPO) promoter (SEQ ID NO:3), for example, and the second nucleic acid sequence can encode a cell toxin, such as Pseudomonas exotoxin A, ricin, or α-sarcin. The second nucleic acid sequence can encode the diphtheria toxin A chain (DT-A), having, for example, the amino acid sequence of SEQ ID NO:2.

Unless otherwise defined, all technical and scientific terms used herein have the same meaning as commonly understood by one of ordinary skill in the art to which this invention pertains. The materials, methods, and examples are illustrative only and not intended to be limiting. Although methods and materials similar or equivalent to those described herein can be used in the practice or testing of the present invention, useful methods and materials are described below. Other features and advantages of the invention will be apparent from the accompanying drawings and description, and from the claims. The contents of all references, pending patent applications and published patents, cited throughout this application are hereby expressly incorporated by reference. In case of conflict, the present specification, including definitions, will control.

### DESCRIPTION OF DRAWINGS

FIG. 1 is the nucleotide sequence of human growth hormone (hGH) (SED ID NO:1) (GenBank Accession Number M13438).

FIG. 2 is the amino acid sequence of diphtheria toxin A chain (SEQ ID NO:2) (GenBank Accession #K01722).

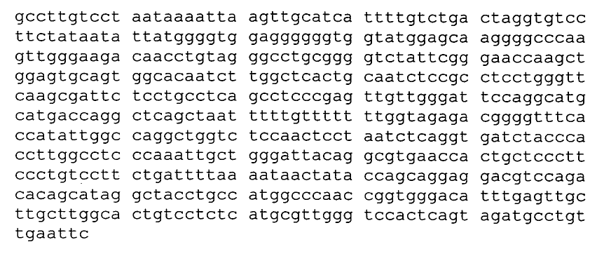

FIG. 3 is the nucleotide sequence of the mouse EPO promoter (SEQ ID NO:3) (GenBank Accession #K01722).

FIG. 4 is a graph showing that eosinophil activation accompanies the pulmonary pathologies occurring in OVA-treated IL-5^{-/-} mice following intratracheal instillation. The graph shows that CD69 expression is limited to eosinophils transferred to OVA-treated mice and does not occur following transfer to naive animals. The graph also shows that the CD69 expression is dependent on CD4⁺ T cells and does not occur in T cell depleted OVA-treated IL-5^{-/-} mice.

FIG. 5 is a graph showing the results of luciferase reporter gene assays. The graph shows that the upstream sequences of the EPO gene confer high level expression (>400-fold) in the human eosinophilic cell line AML14.3D10. PHS1.9-luc is empty vector. MBP4.7-luc is 4.7 kb of sequence upstream from the start site of the MBP-1 gene cloned upstream of the luciferase gene. MBP3.5-luc is 3.5 kb of sequence upstream from the start site of the MPB-2 gene cloned upstream of the luciferase gene. EPO 3.7-luc is 3.7 kb sequence upstream from the start site of the EPO gene cloned upstream of the luciferase gene.

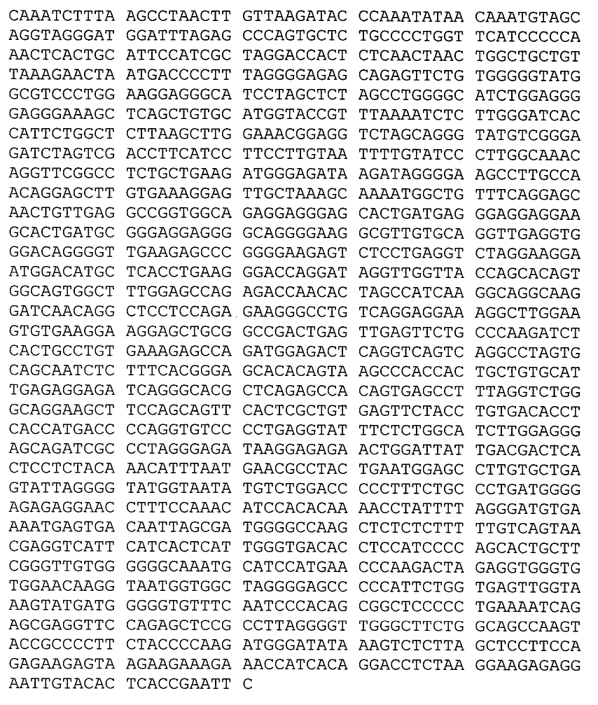

FIG. 6 is an eosinophil lineage-specific transgenic construct mediating Diphtheria Toxin A chain (DT-A) expression using regulatory sequences from the EPO gene.

FIG. 7 is a table with recorded blood cell counts in wildtype (+/+) mice and PHIL mice.

FIG. 8 is an immunohistochemistry stain of bone marrow using anti-MBP polyclonal antisera. The dark spots visible in the wildtype sample are eosinophils. The staining pattern indicates that the bone marrow of the PHIL mouse lacks eosinophils.

FIG. 9A is an immunohistochemistry stain of uterine tissue using anti-MBP polyclonal antisera. The dark spots visible in the wildtype sample are eosinophils. The staining pattern indicates that the uterus of the PHIL mouse lacks eosinophils.

FIG. 9B is an immunohistochemistry stain of tissue from the small intesting of wildtype and PHIL mice. Staining was performed with anti-MBP polyclonal antisera. The dark spots visible in the wildtype sample are eosinophils. The staining pattern indicates that the small intestine of the PHIL mouse lacks eosinophils.

FIG. 9C is an immunohistochemistry stain of thymus tissue using anti-MBP polyclonal antisera. The dark spots visible in the wildtype sample are eosinophils. The staining pattern indicates that the thymus of the PHIL mouse lacks eosinophils.

FIG. 10 is a FACS analysis of peripheral blood from NJ. 1726 homozygous mice and NJ.1726/PHIL mice. Total white blood cells from both strains of mice were stained with an anti-CCR3 PE-conjugated antibody and assessed for the presence of eosinophils (i.e., CCR3+ cells). These data show that while ˜45% of total white blood cells in NJ.1726 mice are eosinophils, this population is absent in NJ.1726/PHIL double transgenic mice.

### DETAILED DESCRIPTION

The features of the invention relate to the development of non-human animal models for the study of physiologies (including pathologies) and treatments relating to tissues and organ systems that typically contain eosinophils. The animal models can express eosinophil-specific transgenes that result in the ablation of eosinophils, but do not significantly affect other hematopoietically-derived cells. Eosinophils are typically located, for example, in the lung, thymus, gut (e.g., intestines), and uterus. The transgenic animal models featured herein can be useful to study the role(s) eosinophils play in pathologies of any or all of these tissues. For example, the animal models featured in the invention can be used to study pulmonary pathologies such as asthma; pathologies of the gut; and/or pathologies of the thymus and T-cell production. The animal models can also be useful for the study of uterine disorders and/or fertility studies.

Eosinophils are granulocytic leukocytes (white blood cells) that originate in bone marrow. Usually small numbers of eosinophils are found in circulation; most eosinophils are found in tissues, such as in the connective tissue immediately underneath the respiratory, gut, and urogenital epithelia. Eosinophils have two kinds of effector function. First, on activation, they release toxic granule proteins and free radicals, which can kill microorganisms and parasites but can also cause significant tissue damage in allergic reactions. Second, activation induces the synthesis of chemical mediators such as prostaglandins, leukotrienes, and cytokines, which amplify the inflammatory response by activating epithelial cells, and recruiting and activating more eosinophils and leukocytes (see Janeway et al., Immunobiology, New York, N.Y.: Garland Publishing, 2001)

Eosinophil-specific gene expression One feature of the invention is a nucleic acid construct containing an eosinophil-specific promoter operably linked to a second heterologous nucleic acid sequence. The heterologous nucleic acid sequence can encode a protein or RNA that causes cell death. Therefore, the expression of the heterologous nucleic acid sequence under the eosinophil-specific promoter can kill eosinophils, but preferably not other cell types. To maximize gene expression in a non-human mammal (e.g., a mouse or rat), at least a fragment of a eukaryotic gene including a series of exons and introns, such as the human growth hormone (hGH) gene, and a polyadenylation signal, can be fused to the 3′ end of the heterologous sequence. The inclusion of exons and introns at the 3′ terminus induces splicing events that facilitate useful levels of gene expression. For example, 2, 3, 4, or more exons (and the intervening introns) can be fused to the heterologous sequence. A series of exons and introns from the human growth hormone (hGH) gene (SEQ ID NO:1), for example, can be fused to the heterologous sequence.

A transgenic mouse described in the published PCT application WO 00/34304 (hereafter, Lee et al.), contained a nucleic acid construct that included an EPO promoter, and a diphtheria toxin A chain coding sequence followed by an SV40 intron and splice sites and a polyadenylation signal sequence. The mutant diphtheria toxin A chain (tox176) was described as being particularly useful as a cell toxin. The tox176 mutation is a G-to-A substitution at nucleotide 383 that results in an amino acid substition of glycine at position 128 with aspartic acid. The transgenic mouse described in Lee et al. was reported to lack eosinophils, but further analysis revealed this mouse to be normal in its production of eosinophils. An alternative sequence described herein encodes a diphtheria toxin A chain having an aspartic acid at amino acid 128. For example, the diphtheria toxin A chain can have the amino acid sequence of SEQ ID NO:2. In addition, it is possible to use eukaryotice splice sites (e.g., splice sites from the human growth hormone gene) to facilitate useful levels of gene expression.

The term “eosinophil-specific promoter” refers to a nucleic acid that provides expression of a nucleic acid transcript preferentially in eosinophils and eosinophil lineage-committed precursors. Typically, such cells are found in the circulation and bone marrow. In mice, these cells also are found in the spleen. An eosinophil-specific promoter can be the promoter of the EPO gene (SEQ ID NO:3).

As used herein, “heterologous” refers to a nucleic acid sequence other than a sequence found adjacent to an eosinophil-specific promoter in vivo. For example, a heterologous sequence can be any sequence other than an eosinophil-specific nucleic acid (e.g., an eosinophil-specific promoter, coding sequence, transcribed sequence, etc.). A heterologous sequence can be a nucleic acid that encodes a protein, such as a cell toxin. As used herein, “operably linked” refers to connection of the promoter and/or other regulatory elements to a nucleic acid sequence in such a way as to permit eosinophil-specific expression of the heterologous nucleic acid sequence. Additional regulatory elements can include, for example, enhancer sequences, response elements or inducible elements. Such constructs can be produced by recombinant DNA technology methods standard in the art.

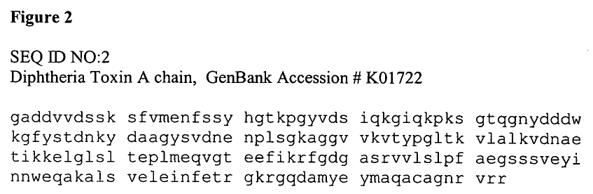

An example of an eosinophil-specific promoter sequence is the EPO promoter shown in FIG. 3 (SEQ ID NO:3) and includes approximately 3770 nucleotides upstream of the EPO gene. An eosinophil-specific promoter can have a nucleotide sequence that deviates from that shown in FIG. 3 and retains the ability to promote eosinophil-specific expression. For example, a nucleic acid sequence can have at least 70% sequence identity to the nucleotide sequence of SEQ ID NO:3 and promote eosinophil-specific expression. In some embodiments, the nucleic acid sequence can have at least 80%, 90%, or 95% sequence identity to SEQ ID NO:3.

Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (B12seq) program from the stand-alone version of BLASTZ containing BLASTN version 2.0.14 and BLASTP version 2.0.14. This stand-alone version of BLASTZ can be obtained from the web site of Fish & Richardson P.C. or from the website of the U.S. government'National Center for Biotechnology Information. Instructions explaining how to use the B12seq program can be found in the readme file accompanying BLASTZ.

B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: −1 is set to a file containing the first nucleic acid sequence to be compared (e.g., C:\seq1.txt); −j is set to a file containing the second nucleic acid sequence to be compared (e.g., C:\seq2.txt); −p is set to blastn; −o is set to any desired file name (e.g., C:\output.txt); −q is set to −1; −r is set to 2; and all other options are left at their default setting. The following command will generate an output file containing a comparison between two sequences: C:\B12seq −i c:\seq1.txt −j c:\seq2.txt −p blastn −o c:\output.txt −q −1 −r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length followed by multiplying the resulting value by 100. For example, if (1) a 2000 nucleotide target sequence is compared to the sequence set forth in SEQ ID NO:3, (2) the B12seq program presents 1031 nucleotides from the target sequence aligned with a region of the sequence set forth in SEQ ID NO:3 where the first and last nucleotides of that 1031 nucleotide region are matches, and (3) the number of matches over those 1031 aligned nucleotides is 850, then the 2000 nucleotide target sequence contains a length of 1031 and a percent identity over that length of 82 (i.e., 850 ) 1031×100=82).

It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

Fragments of the eosinophil-specific promoter can be made that retain the ability to promote eosinophil-specific expression of a nucleic acid sequence of interest (e.g., a nucleic acid that encodes a toxin). Fragments that include the complementary sequence of an eosinophil-specific promoter also can be made. For example, a fragment of an eosinophil-specific promoter can include from about nucleotide 1 to about nucleotide 3710, from about nucleotide 1 to about nucleotide 2565, or from about nucleotide 110 to about nucleotide 2565 or to about nucleotide 3710 of the nucleic acid sequence of SEQ ID NO:3. The ability of fragments to promote eosinophil-specific expression can be assayed using the methods described herein. A fragment of an eosinophil promoter can be used to kill all or some eosinophils; various fragments can exhibit various promoter strengths to achieve various degrees of eosinophil ablation. A fragment can be operably linked to a nucleic acid sequence, such as a sequence encoding a toxin, and used to produce transgenic mice. Eosinophil-specific expression of the gene product encoded by the nucleic acid sequence can be monitored in transgenic mice using standard techniques.

An eosinophil-specific promoter can be cloned from the 5′ flanking sequences of a genomic EPO gene, or can be obtained by other means including chemical synthesis and polymerase chain reaction (PCR) technology using oligonucleotide pairs such as 5′-GGA TCC CCT GGA GCT GGA G-3′ (SEQ ID NO:4) and 5′-GAA TTC GGT GAG TGT ACA ATT CC-3′ (SEQ ID NO:5). PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. PCR is described, for example in PCR Primer: A Laboratory Manual, Ed. by Dieffenbach, C. and Dveksler, G., Cold Spring Harbor Laboratory Press, 1995. Nucleic acids also can be amplified by ligase chain reaction, strand displacement amplification, self-sustained sequence replication or nucleic acid sequence-based amplification. See, for example, Lewis, Genetic Engineering News 12:1, 1992; Guatelli et al., Proc. Natl. Acad. Sci. USA 87:1874-1878, 1990; and Weiss, Science 254:1292, 1991.

The heterologous nucleic acid sequence included in a nucleic acid construct described herein can, for example, encode a protein, an antisense nucleic acid sequence, or a ribozyme. The heterologous nucleic acid sequence can encode a full-length protein, an N- or C-terminal truncation of a full-length protein, or a mutant protein.

For example, the heterologous nucleic acid sequence can encode a cell toxin such as a diphtheria toxin A chain (DT-A) or Pseudomonas exotoxin A. These toxins inactivate elongation factor 2, an essential factor in protein synthesis through ADP-ribosylation. The modifications induced by the toxins occur at a unique post-translational histidine derivative, diphthamide, that is present in the ribosomal binding site of the elongation factor. Another suitable class of cell toxins includes ricin, a plant toxin and a-sarcin, a member of a family of fungal toxins. These toxins inactivate the large ribosomal subunit through hydrolytic alterations of 23-28S RNA. Ricin-type toxins act as specific N-glycosidases, whereas α-sarcin-type toxins act as specific endonucleases (Perentesis et al., Biofactors 3:173-184, 1992). The genes encoding the cytotoxic A chains of diphtheria toxin, Pseudomonas exotoxin, ricin and a-sarcin have been cloned and sequenced (see, for example, Horton et al., J. Leukocyte Biol. 60:285, 1996; Wendt, Gene 124:239-244, 1993; Chen et al., J. Gen. Microbiol. 133:3081-3091, 1987; and Sundan et al., Nucl. Acids Res. 17:1717-1737, 1989).

A heterologous nucleic acid sequence that encodes an antisense nucleic acid sequence or a ribozyme can be cell toxic, such that the population of eosinophils is reduced or abolished.

“Antisense nucleic acid sequences” refers to nucleic acid sequences that are complementary to at least a portion of a target RNA. The term “complementary” refers to a sequence that is able to hybridize with the RNA, forming a stable duplex under normal in vivo conditions. The ability to hybridize depends on both the degree of complementarily and the length of the antisense nucleic acid. Generally, the longer the hybridizing nucleic acid, the more base mismatches with an RNA it may contain and still form a stable duplex. One skilled in the art can ascertain a tolerable degree of mismatch by use of standard procedures to determine the melting point of the hybridized complex.

Antisense nucleic acid sequences can be full-length or less than full-length, and can be complementary to coding or non-coding regions. Antisense nucleic acid sequences that are less than full-length typically are at least six nucleotides in length, and can range from 6 to about 200 nucleotides in length.

“Ribozyme” refers to molecules designed to catalytically cleave targeted RNA transcripts, preventing expression of the protein encoded-by the RNA transcript. Various ribozymes that cleave RNA can be used. For example, hammerhead ribozymes cleave RNAs at locations dictated by flanking regions that form complementary base pairs with the target RNA. The sole requirement is that the target RNA have the following sequence of two bases: 5′-UG-3′. The construction and production of hammerhead ribozymes is known in the art (see, for example, U.S. Pat. No. 5,254,678). Alternatively, RNA endoribonucleases such as the one that occurs naturally in Tetrahymena thermophila can be used (see, for example, U.S. Pat. No. 4,987,071).

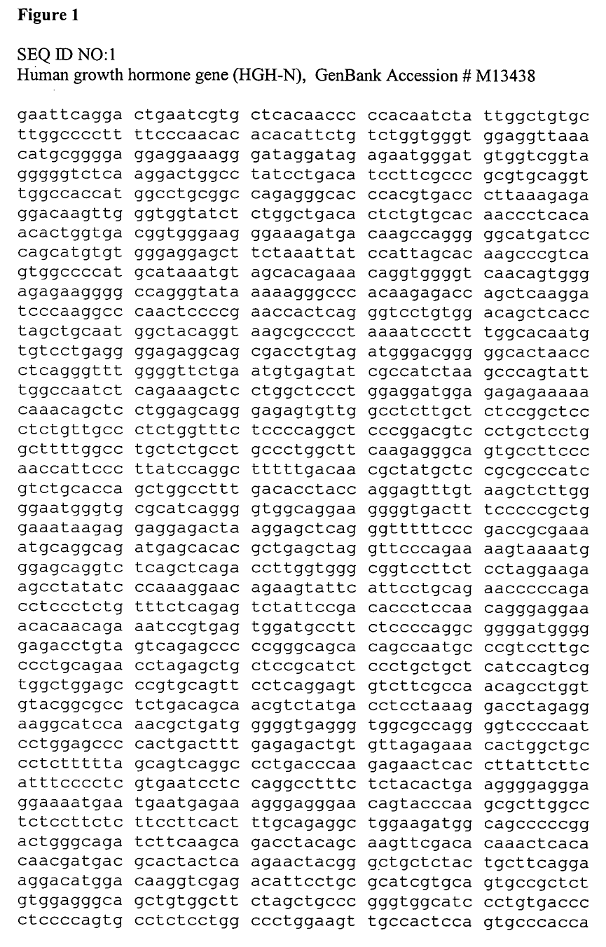

Transgenic Non-Human Mammals Another feature of the invention is a transgenic non-human mammal including a nucleic acid construct, such as any of the constructs described herein. The term “transgenic non-human mammal” includes progeny of the founder transgenic non-human mammal, that retain the nucleic acid construct. The nucleic acid construct includes an eosinophil-specific promoter fragment operably linked to a heterologous nucleic acid sequence, which is operably linked to at least a fragment of a eukaryotic gene including a series of exons and introns, such as the human growth hormone (hGH) gene (FIG. 1). The heterologous nucleic acid sequence is specifically expressed in cells of eosinophil lineage within the transgenic non-human mammal. The heterologous nucleic acid sequence can be a gene encoding a protein or protein fragment, an antisense nucleic acid sequence, or a ribozyme. For example, the heterologous nucleic acid sequence can be a cell toxin gene (or fragment thereof) such as the DT-A gene encoding the diphtheria toxin. To maximize gene expression, a fragment of a eukaryotic gene, including a series of exons and introns, such as a fragment of the human growth hormone gene, and a polyadenylation signal can be fused to the 3′ end of the heterologous sequence.

A useful feature of the transgenic non-human mammals is that cells other than those of the eosinophilic lineage are generally unaffected by activity of the transgene. As such, the transgenic mammals typically have normal levels of red blood cells and other hematopoietic cells. Transgenic non-human mammals can be farm animals such as pigs, goats, sheep, cows, horses and rabbits, rodents such as rats, guinea pigs and mice, and non-human primates such as baboons, monkeys and chimpanzees. Transgenic mice are particularly useful.

Various techniques known in the art can be used to introduce nucleic acid constructs into non-human mammals to produce the founder lines of the transgenic non-human mammals. Such techniques include, but are not limited to, pronuclear microinjection (U.S. Pat. No. 4,873,191), retrovirus mediated gene transfer into germ lines (Van der Putten et al., Proc. Natl. Acad. Sci. USA 82:6148, 1985), gene targeting into embryonic stem cells (Thompson et al., Cell 56:313, 1989), electroporation of embryos (Lo, Mol. Cell. Biol. 3:1803, 1983), and transformation of somatic cells in vitro followed by nuclear transplantation (Wilmut et al., Nature 385:810-813, 1997).

Once transgenic non-human mammals have been generated, eosinophil-specific expression of the second nucleic acid sequence can be assessed using standard techniques. Initial screening can be accomplished by Southern blot analysis or PCR techniques to determine whether or not integration of the transgene has taken place. The level of mRNA expression of the second nucleic acid sequence in the tissues of the transgenic non-human mammals can be assessed using techniques that include, but are not limited to, Northern blot analysis of tissue samples obtained from the animal, in situ hybridization analysis and reverse-transcriptase PCR (RT-PCR). Standard histochemical and immunohistochemical techniques also can be used to assess the presence or absence of eosinophils in transgenic non-human mammals expressing a cell toxin.

Antigen-induced mouse models of pulmonary allergic disease have proven particularly informative in the dissection of inflammatory pathways in the lung. Typically, these models involve sensitization with a specific pulmonary effector (e.g., an antigen such as ovalbumin (OVA)) followed by airborne administration of the same antigen (Blyth et al., Am. J. Respir. Cell Mol. Biol. 14:425-438, 1996). Sensitized mice treated with aerosolized allergen develop leukocytic infiltrates of the airway lumen dominated by CD4⁺ lymphocytes and eosinophils. These mice also develop many of the changes pathognomonic of asthma including AHR and goblet cell hyperplasia with excessive mucus production.

In general, OVA-induced type I hypersensitivity in the lung shares many characteristics with human atopic asthma, although specific issues influence conclusions drawn from these models: (i) Immunologic differences exist between mice and humans. For example, while antigen-mediated airway responses in the mouse can be mediated by IgG₁, it appears likely that human responses are restricted to IgE pathways. This difference is reflected in observations that mouse eosinophils lack FcεRII and FcεRI, the low and high-affinity IgE receptors, respectively; (ii) Results from OVA mouse models vary based on the protocol used and the genetic strain of the experimental animals. Choices for both parameters are often based on predetermined experimental endpoints, e.g., some strains of mice display significantly greater levels of AHR and some protocols maximize and/or minimize the roles of IgE and mast cells.

Despite these differences, OVA models have been productively used to examine the details of the underlying molecular and cellular events associated with pulmonary inflammation. These studies have demonstrated that inflammatory pathologies of the lung are dependent on both T cell dependent eosinophil-mediated effector functions and immunoglobulin/mast cell dependent pathways. The relative importance of each pathway, as well as the interactions between them, are not fully understood. Moreover, eosinophil-mediated effector functions still remain a vague description of activities that are apparently critical to the onset/progression of pulmonary pathology.

Utilizing the expression of toxic gene products permits the eosinophil lineage to be genetically ablated within the transgenic non-human mammal. Thus, otherwise normal non-human mammals substantially free of the eosinophil lineage can be generated, allowing the role(s) of the eosinophil in the onset/progression of, for example, allergic pulmonary pathology to be addressed. The absence of eosinophils may not affect animal survival, although the loss of peripheral eosinophils can limit the development of tissue pathologies (e.g., pulmonary pathologies).

Transgenic mice that are substantially free of eosinophils can be sensitized with OVA and then challenged with OVA to induce histopathological changes. For example, alum-precipitated OVA can be injected intraperitoneally on day 0 and on day 14. On days 24, 25 and 26, the transgenic mice can be challenged with an aerosol of 1% OVA in saline, such as by 20 minute inhalations. OVA-induced histopathologic pulmonary changes can be assessed using standard histological and pulmonary function techniques and compared with non-transgenic OVA sensitized/challenged mice. For example, leukocyte accumulation in the airways and lung tissue can be assessed on days 26, 27, and 28 by bronchoalveolar lavage (BAL) and immunohistochemistry of lung tissue samples. Leukocytes can be harvested, for example, from peripheral blood, such as from the tail vasculature, and from femoral bone marrow. The effect of OVA challenge can also be examined by histology studies of the lung using stains such as hematoxylin-eosin (H & E), Masson'Trichrome, Alcian Blue, and Periodic Acid Schiff (PAS). Eosinophil-specific antibodies can also be used. The effect of other allergens, such as ragweed, on lung morphology and leukocyte accumulation can also be tested.

Eosinophil-deficient mice can also be used to investigate the role of eosinophil effector functions (EEFs). For example, test populations of eosinophils (such as IL-13 deficient eosinophils) can be injected into OVA sensitized/aerosol challenged eosinophil-deficient mice. Characterization of pulmonary pathologies compared to mice injected with IL-13⁺ eosinophils can provide insight to the role of IL-13 in an allergen response.

Changes in the lung immune microenvironment such as an inflammatory response, T_{H}1and T_{H}2 responses, eosinophil-induced neutrophil recruitment, and modulation of T_{H}1/T_{H}2 lymphocyte subtypes can be determined by measuring cytokine levels in brochoalveolar lavage (BAL) fluid. For example, levels of INF-γ, TGFβ, IL-4, IL-5, IL-.6, and/or IL-13 can be determined. Immunocytochemistry techniques (such as ELISA) can be used to measure cytokine levels.

The transgenic mice can be crossed to any number of genetic backgrounds to generate a mouse line that is appropriate for any intended purpose. For example, eosinophil-deficient mice can be crossed into a BALB/c genetic background. BALB/c mice are less sensitive to the loss of IL-5. Eosinophil-deficient mice can be crossed to other engineered lines, such as a strain that exhibits eosinophilia. For example, a strain that expresses IL-5 constitutively, such as NJ.1726, exhibits eosinophilia. Progeny from an NJ.1726 mouse crossed with an eosinophil-deficient transgenic mouse as described herein, will continue to constitutively express IL-5, but the strain will not produce eosinophils. This strain of mice can provide information regarding the different effects of eosinophilia and IL-5 overproduction, and other secondary effects on lung pathologies.

Eosinophil-deficient transgenic mice can be used to examine late phase bronchoconstriction following allergen provocation. For example, OVA-challenged mice as described above, can be further challenged with an OVA aerosol (e.g., 5% in saline), such as on Day 28 (see above). Late phase bronchoconstriction can be assayed by measuring inspiratory/expiratory flow using techniques such as whole-body plethysmography or a forced ventilator system.

The eosinophil-deficient transgenic mice can be used to investigate the physiology of any tissue or organ where eosinophils are located (e.g., lung, uterus, intestines, and thymus). For example, the mice are useful for the investigation of uterine disorders such as disorders resulting in infertility or low fertility. Eosinophil-deficient mice can also be useful for the investigation of gut disorders, including disorders of the intestines, or for disorders of the thymus.

The invention is further illustrated by the following examples, which should not be construed as further limiting.

### EXAMPLES

### Example 1

### Adoptive Transfer of Eosinophils into IL-5^{-/-} Mice Demonstrates their Causative Link to Allergen-Mediated Pulmonary Pathologies

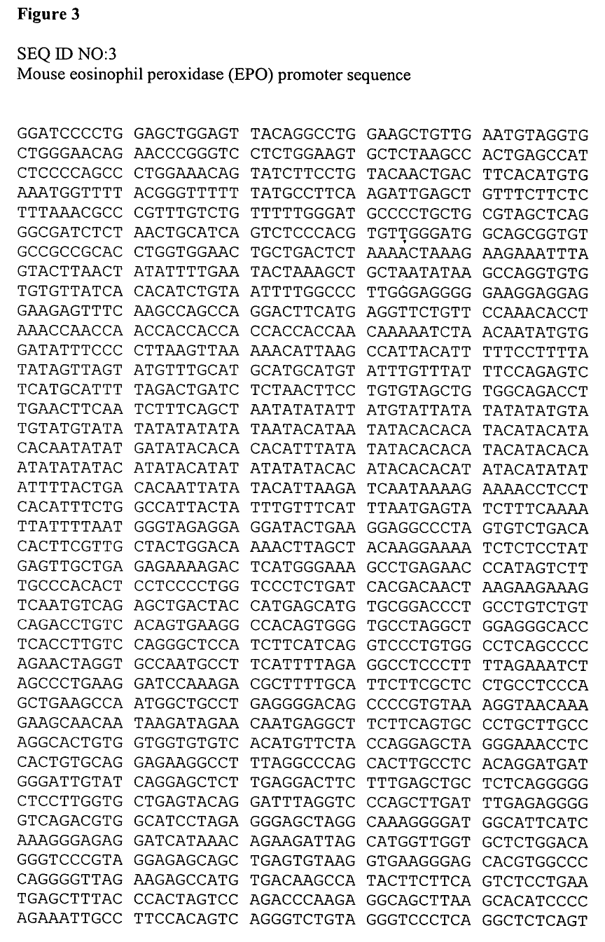

Eosinophils were transferred directly into the lungs of either naïve or ovalbumin (OVA)-treated IL-5^{-/-} mice (i.e., animals with low numbers of eosinophils owing to the absence of the prominent factor required for population expansion). This strategy resulted in a pulmonary eosinophilia in IL-5^{-/-} mice equivalent to that observed in OVA-treated wildtype animals. A concomitant consequence of this eosinophil transfer was an increase in Th2 Bronchoalveolar lavage (BAL) cytokine levels and the restoration of intracellular epithelial mucus in OVA-treated IL-5^{-/-} mice equivalent to OVA-treated wild type levels. Moreover, the transfer also resulted in the development of airway hyper-responsiveness (AHR), and these pulmonary changes did not occur when eosinophils were transferred into naive IL-5^{-/-} mice, eliminating non-specific consequences of the eosinophil transfer. Significantly, administration of OVA-treated IL-5^{-/-} mice with GK1.5 (anti-CD4) antibodies abolished the increases in mucus accumulation and AHR following transfer of eosinophils. The development of pathologies in OVA-treated, but not saline control, mice was associated with CD69 expression on the transferred eosinophils. Moreover, this CD69 expression on eosinophils was T cell dependent and did not occur in mice depleted of CD4⁺ T cells (FIG. 4). Thus, CD4⁺ T cells, as well as eosinophils, are each necessary, yet alone insufficient, for the development of allergic pulmonary pathology.

These data provide evidence that the perturbations of lung parameters associated with mouse models which manipulate IL-5 levels are a consequence of effects on pulmonary eosinophil numbers and, in turn, EEFs. These data also support an expanded view of eosinophil activities in the lung and suggest that interactions with T cells are underlying mechanisms leading to allergic respiratory inflammation and lung dysfunction. In addition, the development of a viable strategy to isolate and adoptively transfer eosinophils provides, together with a uniquely eosinophil-less recipient mouse (see examples below), a model system to define the role(s) of eosinophils in the development of allergen-mediated pulmonary pathologies.

### Example 2

### The Ablation of Eosinophils using a Depleting Antibody Leads to the Loss and/or Attenuation of Allergen-Mediated Pulmonary Pathologies

To examine the relationship between allergen-induced pulmonary eosinophilia and the onset/progression of lung pathologies, a strategy to deplete eosinophils from the lungs of allergen sensitized/challenged mice was developed. Concurrent administration of a rat anti-mouse CCR3 monoclonal antibody (Grimaldi et al., J. Leukoc. Biol. 65:846, 1999) into the peritoneal cavity (systemic) and as an aerosol to the lung (local), resulted in the near abolition of eosinophils from the lung. The airway lumen of antibody-treated OVA-challenged mice was essentially devoid of eosinophils. Moreover, perivascular/peribronchial eosinophil levels in these mice were reduced to levels indistinguishable from naive saline challenged animals. This antibody-mediated depletion did not affect any other leukocyte population, nor did this dual systemic-local strategy affect other allergic inflammatory responses. In particular, neither OVA-specific immunoglobulin production (i.e., B cell activities) nor T cell dependent elaboration of Th2 cytokines were altered. Moreover, unlike reports of allergen-treated CCR3^{-/-} mice (Humbles et al., Proc. Natl. Acad. Sci. USA 99:1479, 2002), depletion of eosinophils using this anti-CCR3 approach neither ablated nor changed the number of lung tissue mast cells in OVA-treated animals. The unique ablation of virtually all pulmonary eosinophils, without concurrent effects on other leukocyte populations/activities, resulted in a significant decrease in airway mucus production and abolished allergen-induced airway hyperresponsiveness.

These data demonstrate a direct causative relationship between allergen-mediated pathologies and the presence of eosinophils. Eosinophils were shown to contribute to both pulmonary histopathology (e.g., mucus accumulation) and lung dysfunction (e.g., AHR). However, this model does not differentiate between effects on eosinophils and other CCR3⁺0 cells and thus, the data are equivocal. Moreover, the hegemonic role of CD4+T cells suggests that any of the observed effects on eosinophil activities are a consequence of T cell induced eosinophil activation. These preliminary successes emphasize the importance of developing a strain of mice congenitally devoid of eosinophils (see below) to better define the role(s) of eosinophils during allergen-mediated inflammatory responses in the lung.

### Example 3

### A Promoter Fragment can Elicit Eosinophil-Specific Gene Expression in Transgenic Mice

We have cloned and characterized genes encoding abundant eosinophil secondary granule proteins (ESGPs) in the mouse, including MBP-1 (Larson et al., J. Immunol. 155:3002, 1995), MBP-2 (Macias et al., Jour Leukocyte Biol. 67:567, 2000), and EPO (Horton et al., J. Leukocyte Biol. 60:285, 1996). This sequence information is inclusive of nearly 5 kb of DNA upstream of the transcription start sites. Sequence analyses/alignments did not reveal extensive regions of identity between the “promoters” of these genes, but several transcription factor binding sites were conserved, including sites for the basal transcription apparatus (e.g., TFIID, SP1). Also present were binding sites for transcription factors that have been shown to bind human ESGP gene promoters (e.g., GATA-1). We used a human eosinophil-committed cell line to test the efficacy of the promoter fragments. Conservation of the ESGP genes between mouse and humans (Larson et al., J. Immunol. 155:3002, 1995; Macias et al., Jour. Leukocyte Biol. 67:567, 2000; Horton et al., J. Leukocyte Biol. 60:285, 1996; Larson et al., Proc. Natl. Acad. Sci. USA 93:12370, 1996) supported the viability of this approach.

Representative data from transfections of promoter-luciferase reporter constructs into AML14.3D10 cells (Paul et al., J. Leukocyte Biol. 56:74, 1994) are shown in FIG5. The 4.7 kb upstream MBP-1 sequences support expression above background in these cells. The increase, although significant, was nominal. Similar results were observed using 3.5 kb of upstream sequences from the MBP-2 gene. However, a 3.8 kb fragment containing upstream sequences from the mouse EPO gene elicited a nearly 400-fold increase in luciferase expression, indicating that this fragment is a potential candidate for the generation of transgenic mice.

The data demonstrated that the upstream sequences from several ESGP genes will support cell-specific gene expression. In particular, these in vitro studies suggested that the mouse EPO promoter fragment will be most suitable to drive high-level expression in eosinophil lineage committed cells. These data have led us to the development of a transgenic construct and, in turn, to a line of mice in which eosinophils are eliminated through lineage restricted expression of the suicide gene, Diphtheria Toxin A (DT-A).

### Example 4

### A Transgenic Line of Mice (Line: “PHIL”) Devoid of Eosinophils was Generated Through Eosinophil Lineage-Specific Expression of DT-A

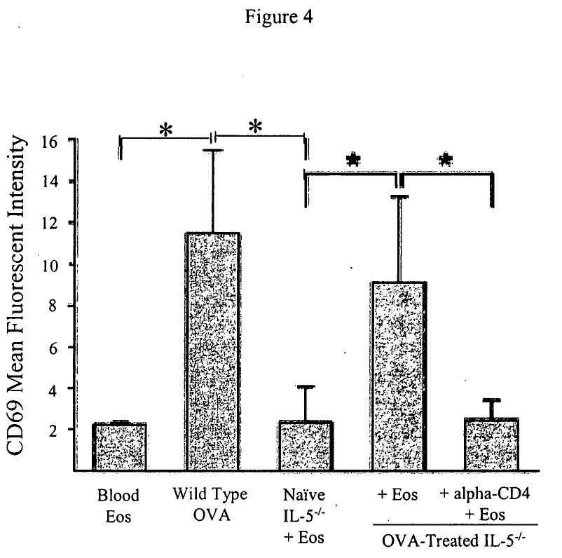

A transgenic construct was developed (FIG. 6) using mouse EPO-derived sequences in conjunction with the DT-A open reading frame (Palmiter et al., Cell 50:435, 1987 (published erratum appears in Cell 63:following 608, 1990); Breitman et al., Science 238:1563, 1987) and a series of exons/introns derived from the human growth hormone gene to provide the splicing events required for high-level expression (Brinster et al., Proc. Natl. Acad. Sci. USA 85:836, 1988; Palmiter et al., Proc. Natl. Acad. Sci. USA 88:478, 1991).

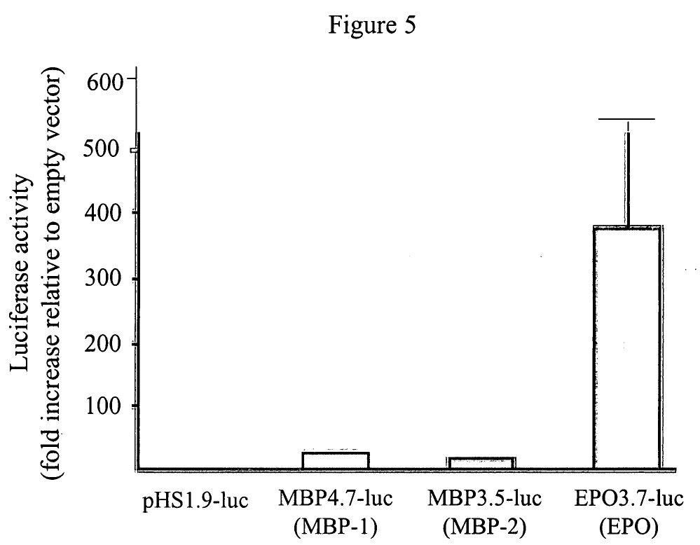

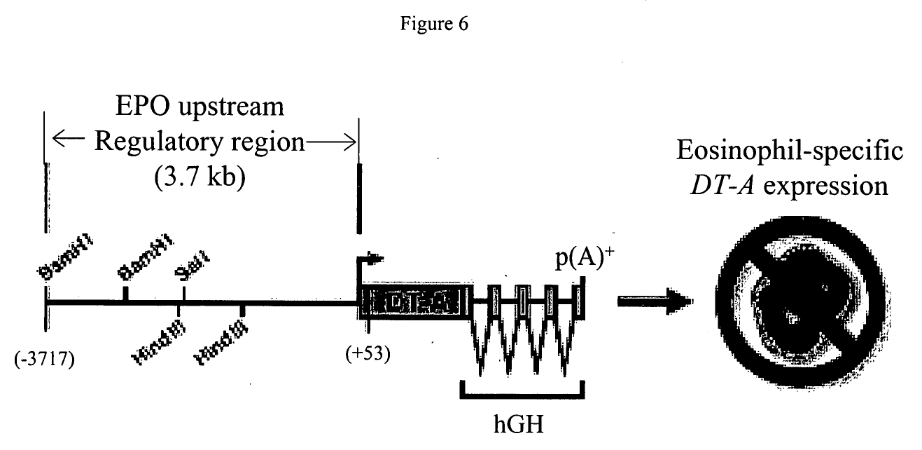

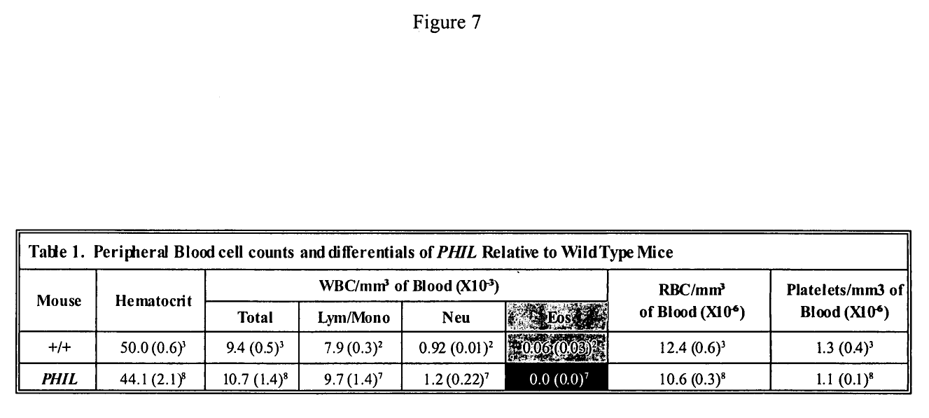

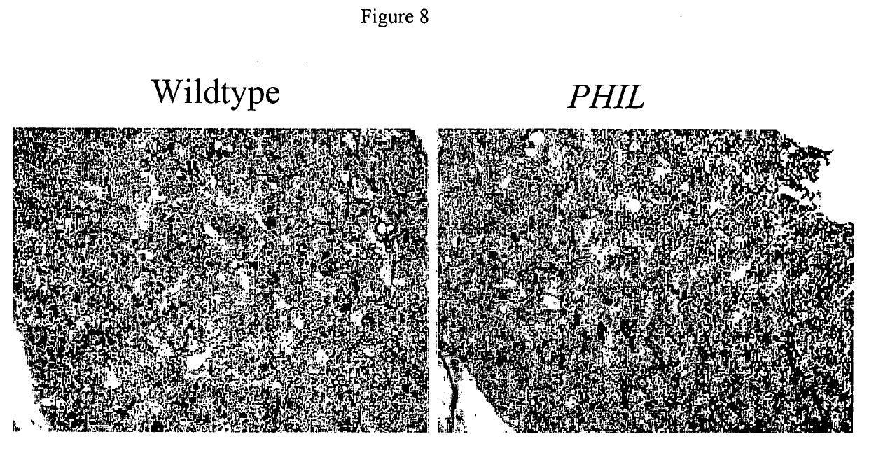

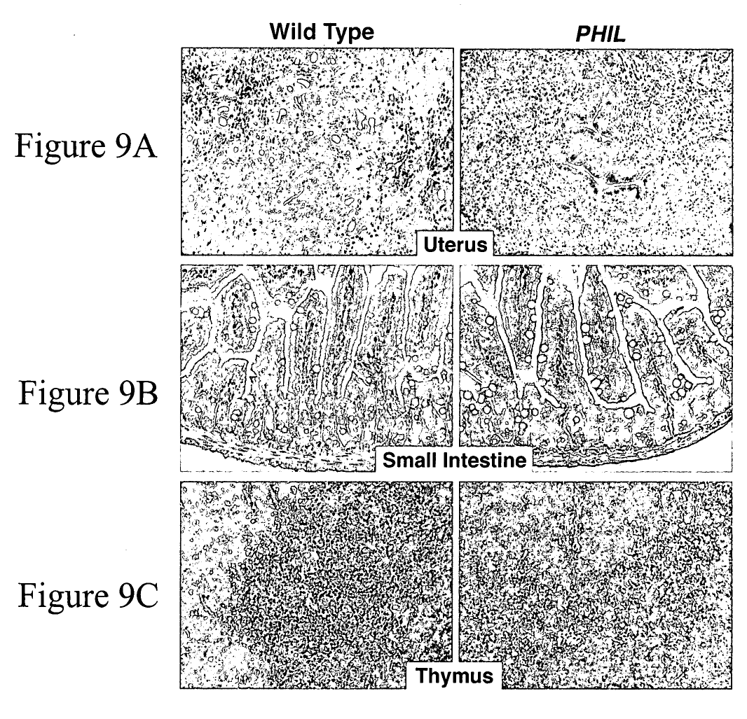

Assessments of circulating leukocytes and other hematopoietic parameters demonstrated that these transgenic mice are devoid of eosinophils without any effects on other cell types (FIG. 7). This eosinophil deficiency is life-long and is a Mendelian inheritable trait of the line (four successive generations of mice examined to date). The eosinophil-deficient character of PHIL mice extends, as expected, to sites of hematopoiesis such as spleen and bone marrow, where cell differentials and immunohistochemistry with antibodies specific for MBP show that these compartments are devoid of eosinophils with no effects on other cell populations (FIG. 8). In addition, an examination of all tissues with abundant resident populations of eosinophils in wild type animals (i.e., uterus, intestines, and thymus) demonstrated that each are devoid of these cells (FIGS. 9A, 9B, and 9C). Moreover, assessments of both lung sections and peritoneal cavity cells showed that in addition to the absence of effects on circulating leukocytes, mast cell numbers were also unaffected in PHIL mice.

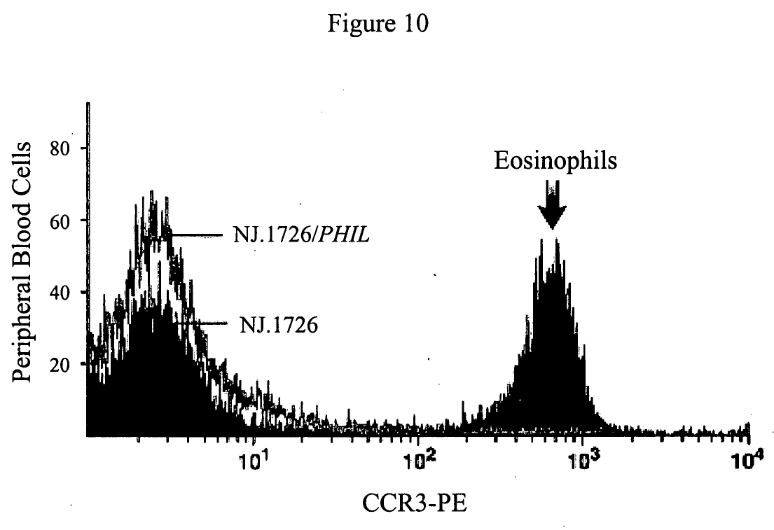

To demonstrate that these animals are truly eosinophil deficient, mice were crossed with multiple IL-5 transgenic lines (Lee et al., J. Immunol. 158:1332, 1997; Lee et al., J. Exp. Med. 185:2143, 1997) that, in some cases, had circulating eosinophil levels in excess of 100,000 eosinophils/mm³, representing >40% of total white blood cells. Significantly, FACS analyses and cell differentials of blood from these double transgenic animals showed that despite the eosinophilopoietic effects of IL-5 overexpression, in the presence of the DT-A transgene the double transgenic animals remained devoid of eosinophils (FIG. 10). This remarkable ablation of all eosinophils in double transgenic mice also extended to the spleen and the bone marrow.

The line of mice described herein is uniquely devoid eosinophils. An eosinophil-deficient line of mice developed by other investigators (Yu et al., J. Exp. Med. 195:1387, 2002) has secondary effects leading to defects in erythropoiesis. These defects include a nearly 30% decrease in circulating red blood cells. The lack of any such secondary effects in our new transgenic line makes these mice a unique and novel model with which to test the role(s) of eosinophils in models of human disease. For example, the animals can be used to assess (1) eosinophil contributions to pathologies arising in a acute allergen sensitization/aerosol challenge model; (2) eosinophil contributions to lung remodeling in a chronic exposure model; (3) the specific role(s) of eosinophils in the late phase reaction following allergen-provocation; and (4) the role(s) of these cell in the pathologies arising in the lungs IL-5 transgenic models of asthma.

### Example 5

### Female PHIL Mice have Decreased Fecundity

Wildtype female mice and PHIL female mice were bred to age-matched wildtype males. Of the wildtype female/male matings, nine females demonstrated copulatory plugs, and of these, seven (78%) delivered pups. Of the PHIL female/wildtype male matings, 13 females demonstrated copulatory plugs, and only two of the 13 (15%) delivered pups.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

1. A transgenic non-human mammal comprising a nucleic acid construct, said nucleic acid construct comprising a first nucleic acid sequence operably linked to a second nucleic acid sequence, wherein said second nucleic acid sequence is operably linked to a third nucleic acid sequence, wherein said first nucleic acid sequence promotes eosinophil-specific expression of said second nucleic acid sequence, said second nucleic-acid sequence encodes a toxin, and said third nucleic acid sequence comprises a sequence from a human Growth Hormone (hGH) gene; and wherein said non-human mammal is substantially free of eosinophils, and said non-human mammal has a substantially normal level of red blood cells.

2. The transgenic non-human mammal of claim 1, wherein said non-human mammal is a rodent.

3. The transgenic non-human mammal of claim 1, wherein said rodent is a mouse.

4. The transgenic non-human mammal of claim 1, wherein said second nucleic acid sequence encodes Diphtheria toxin A chain (DT-A).

5. The transgenic non-human mammal of claim 1, wherein said second nucleic acid sequence encodes the amino acid sequence of SEQ ID NO:2.

6. The transgenic non-human mammal of claim 1, wherein said second nucleic acid sequence encodes Pseudomonas exotoxin A.

7. The transgenic non-human mammal of claim 1, wherein said second nucleic acid sequence encodes ricin.

8. The transgenic non-human mammal of claim 1, wherein said second nucleic acid sequence encodes α-sarcin.

9. The transgenic non-human mammal of claim 1, wherein said first nucleic acid sequence comprises at least a fragment of the sequence of SEQ ID NO:3.

10. A method for investigating a role for eosinophils in pulmonary physiology, comprising:
(i) providing a transgenic non-human mammal of claim 1;
(ii) exposing said transgenic non-human mammal to a pulmonary effector;
(iii) comparing lung tissue from said exposed transgenic non-human mammal to lung tissue from a control non-human mammal; and
(iv) identifying a role, or a potential role, of eosinophils in pulmonary physiology based, at least in part, on said comparison.

11. The method of claim 10, wherein said pulmonary effector is an allergen.

12. The method of claim 10, wherein said control non-human mammal is a non-transgenic non-human mammal exposed to said pulmonary effector.

13. The method of claim 10, wherein said control non-human mammal is a non-transgenic non-human mammal, not exposed to said pulmonary effector.

14. The method of claim 10, wherein said control non-human mammal is a transgenic non-human mammal of claim 1, not exposed to said pulmonary effector.

15. A method for investigating a role for eosinophils in uterine physiology, comprising:
(i) providing a transgenic non-human mammal of claim 1;
(ii) exposing said transgenic non-human mammal to a test compound;
(iii) comparing uterine tissue from said exposed transgenic non-human mammal to uterine tissue from a control non-human mammal; and
(iv) identifying a role, or a potential role, of eosinophils in uterine physiology based, at least in part, on said comparison.

16. The method of claim 15, wherein said control non-human mammal is a non-transgenic non-human mammal exposed to said test compound.

17. The method of claim 15, wherein said control non-human mammal is a non-transgenic non-human mammal, not exposed to said test compound.

18. The method of claim 15, wherein said control non-human mammal is a transgenic non-human mammal of claim 1, not exposed to said test compound.

19. A method for investigating a role for eosinophils in intestine physiology, comprising:
(i) providing a transgenic non-human mammal of claim 1;
(ii) exposing said transgenic non-human mammal to a test compound;
(iii) comparing intestinal tissue from said exposed transgenic non-human mammal to intestinal tissue from a control non-human mammal; and
(iv) identifying a role, or a potential role, of eosinophils in intestinal physiology based, at least in part, on said comparison.

20. The method of claim 19, wherein said control non-human mammal is a non-transgenic non-human mammal exposed to said test compound.

21. The method of claim 19, wherein said control non-human mammal is a non-transgenic non-human mammal, not exposed to said test compound.

22. The method of claim 19, wherein said control non-human mammal is a transgenic non-human mammal of claim 1, not exposed to said test compound.

23. A method for investigating a role for eosinophils in thymus physiology, comprising:
(i) providing a transgenic non-human mammal of claim 1;
(ii) exposing said transgenic non-human mammal to a test compound;
(iii) comparing thymus tissue from said exposed transgenic non-human mammal to thymus tissue from a control non-human mammal; and
(iv) identifying a role, or a potential role, of eosinophils in thymus physiology based, at least in part, on said comparison.

24. The method of claim 23, wherein said control non-human mammal is a non-transgenic non-human mammal exposed to said test compound.

25. The method of claim 23, wherein said control non-human mammal is a non-transgenic non-human mammal, not exposed to said test compound.

26. The method of claim 23, wherein said control non-human mammal is a transgenic non-human mammal of claim 1, not exposed to said test compound.

27. A method of classifying a test compound as a positive or negative drug candidate, the method comprising:
(i) contacting a transgenic non-human mammal of claim 1 with a test compound;
(ii) examining an organ or tissue of said contacted transgenic non-human mammal for a presence, absence, or degree of physiological change in said organ or tissue; and
(iii) classifying said test compound as a positive or negative drug candidate based on said presence, absence, or degree of said physiological change.

28. The method of claim 27, wherein said organ or tissue is lung tissue.

29. The method of claim 27, wherein said organ or tissue is the gut.

30. The method of claim 27, wherein said organ or tissue is the thymus.

31. The method of claim 27, wherein said organ or tissue is the uterus.

32. A nucleic acid construct comprising a first nucleic acid sequence operably linked to a second nucleic acid sequence heterologous to said first nucleic acid sequence, wherein said first nucleic acid sequence promotes eosinophil-specific expression of said second nucleic acid sequence, and wherein said second nucleic acid sequence is operably linked to at least a fragment of a human growth hormone gene.

33. The nucleic acid construct of claim 32, wherein said first nucleic acid sequence comprises the sequence of SEQ ID NO:3.

34. The nucleic acid construct of claim 32, wherein said second nucleic acid sequence encodes a cell toxin.

35. The nucleic acid construct of claim 32, wherein said second nucleic acid sequence encodes a diphtheria toxin A chain (DT-A).

36. The nucleic acid construct of claim 32, wherein said second nucleic acid sequence encodes the amino acid sequence of SEQ ID NO:2.

37. The nucleic acid construct of claim 32, wherein said second nucleic acid sequence encodes Pseudomonas exotoxin A.

38-39. (canceled)

40. The transgenic non-human mammal of claim 1, wherein said sequence from said hGH gene is at least a fragment of the sequence of SEQ ID NO:1.

41. The transgenic non-human mammal of claim 1, wherein said sequence from said hGH gene comprises at least two exons and at least one intron.


SEQUENCE LISTING (5 sequences)

SEQ ID NO: 1 - DNA, 2657 bp, Homo sapiens
gaattcaggactgaatcgtgctcacaacccccacaatctattggctgtgcttggccccttttcccaacacacacattctgtctggtgggtggaggttaaacatgcggggaggaggaaagggataggatagagaatgggatgtggtcggtagggggtctcaaggactggcctatcctgacatccttcgcccgcgtgcaggttggccaccatggcctgcggccagagggcacccacgtgacccttaaagagaggacaagttgggtggtatctctggctgacactctgtgcacaaccctcacaacactggtgacggtgggaagggaaagatgacaagccagggggcatgatcccagcatgtgtgggaggagcttctaaattatccattagcacaagcccgtcagtggccccatgcataaatgtagcacagaaacaggtggggtcaacagtgggagagaaggggccagggtataaaaagggcccacaagagaccagctcaaggatcccaaggcccaactccccgaaccactcagggtcctgtggacagctcacctagctgcaatggctacaggtaagcgcccctaaaatccctttggcacaatgtgtcctgaggggagaggcagcgacctgtagatgggacgggggcactaaccctcagggtttggggttctgaatgtgagtatcgccatctaagcccagtatttggccaatctcagaaagctcctggctccctggaggatggagagagaaaaacaaacagctcctggagcagggagagtgttggcctcttgctctccggctccctctgttgccctctggtttctccccaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgtaagctcttggggaatgggtgcgcatcaggggtggcaggaaggggtgactttcccccgctggaaataagaggaggagactaaggagctcagggtttttcccgaccgcgaaaatgcaggcagatgagcacacgctgagctaggttcccagaaaagtaaaatgggagcaggtctcagctcagaccttggtgggcggtccttctcctaggaagaagcctatatcccaaaggaacagaagtattcattcctgcagaacccccagacctccctctgtttctcagagtctattccgacaccctccaacagggaggaaacacaacagaaatccgtgagtggatgccttctccccaggcggggatgggggagacctgtagtcagagcccccgggcagcacagccaatgcccgtccttgcccctgcagaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatgggggtgagggtggcgccaggggtccccaatcctggagccccactgactttgagagactgtgttagagaaacactggctgccctctttttagcagtcaggccctgacccaagagaactcaccttattcttcatttcccctcgtgaatcctccaggcctttctctacactgaaggggagggaggaaaatgaatgaatgagaaagggagggaacagtacccaagcgcttggcctctccttctcttccttcactttgcagaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagacaacctgtagggcctgcggggtctattcgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctgccatggcccaaccggtgggacatttgagttgcttgcttggcactgtcctctcatgcgttgggtccactcagtagatgcctgttgaattc

SEQ ID NO: 2 - protein, 193 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRR

SEQ ID NO: 3 - DNA, 3771 bp, Mus musculus
ggatcccctggagctggagttacaggcctggaagctgttgaatgtaggtgctgggaacagaacccgggtcctctggaagtgctctaagccactgagccatctccccagccctggaaacagtatcttcctgtacaactgacttcacatgtgaaatggttttacgggttttttatgccttcaagattgagctgtttcttctctttaaacgcccgtttgtctgtttttgggatgcccctgctgcgtagctcagggcgatctctaactgcatcagtctcccacgtgttgggatggcagcggtgtgccgccgcacctggtggaactgctgactctaaaactaaagaagaaatttagtacttaactatattttgaatactaaagctgctaatataagccaggtgtgtgtgttatcacacatctgtaattttggcccttgggagggggaaggaggaggaagagtttcaagccagccaggacttcatgaggttctgttccaaacacctaaaccaaccaaccaccaccaccaccaccaacaaaaatctaacaatatgtggatatttccccttaagttaaaaacattaagccattacatttttccttttatatagttagtatgtttgcatgcatgcatgtatttgtttatttccagagtctcatgcattttagactgatctctaacttcctgtgtagctgtggcagaccttgaacttcaatctttcagctaatatatattatgtattatatatatatgtatgtatgtatatatatatatataatacataatatacacacatacatacatacacaatatatgatatacacacacatttatatatacacacatacatacacaatatatatacatatacatatatatatacacatacacacatatacatatatattttactgacacaattatatacattaagatcaataaaagaaaacctcctcacatttctggccattactatttgtttcatttaatgagtatctttcaaaattattttaatgggtagaggaggatactgaaggaggccctagtgtctgacacacttcgttgctactggacaaaacttagctacaaggaaaatctctcctatgagttgctgagagaaaagactcatgggaaagcctgagaacccatagtctttgcccacactcctcccctggtccctctgatcacgacaactaagaagaaagtcaatgtcagagctgactaccatgagcatgtgcggaccctgcctgtctgtcagacctgtcacagtgaaggccacagtgggtgcctaggctggagggcacctcaccttgtccagggctccatcttcatcaggtccctgtggcctcagccccagaactaggtgccaatgccttcattttagaggcctcccttttagaaatctagccctgaaggatccaaagacgcttttgcattcttcgctcctgcctcccagctgaagccaatggctgcctgaggggacagccccgtgtaaaggtaacaaagaagcaacaataagatagaacaatgaggcttcttcagtgccctgcttgccaggcactgtggtggtgtgtcacatgttctaccaggagctagggaaacctccactgtgcaggagaaggcctttaggcccagcacttgcctcacaggatgatgggattgtatcaggagctcttgaggacttctttgagctgctctcagggggctccttggtgctgagtacaggatttaggtcccagcttgatttgagagggggtcagacgtggcatcctagagggagctaggcaaaggggatggcattcatcaaagggagaggatcataaacagaagattagcatggttggtgctctggacagggtcccgtaggagagcagctgagtgtaaggtgaagggagcacgtggccccaggggttagaagagccatgtgacaagccatacttcttcagtctcctgaatgagctttacccactagtccagacccaagaggcagcttaagcacatccccagaaattgccttccacagtcagggtctgtagggtccctcaggctctcagtcaaatctttaagcctaacttgttaagatacccaaatataacaaatgtagcaggtagggatggatttagagcccagtgctctgcccctggttcatcccccaaactcactgcattccatcgctaggaccactctcaactaactggctgctgttaaagaactaatgacccctttaggggagagcagagttctgtgggggtatggcgtccctggaaggagggcatcctagctctagcctggggcatctggaggggagggaaagctcagctgtgcatggtaccgtttaaaatctcttgggatcaccattctggctcttaagcttggaaacggaggtctagcagggtatgtcgggagatctagtcgaccttcatccttccttgtaattttgtatcccttggcaaacaggttcggcctctgctgaagatgggagataagataggggaagccttgccaacaggagcttgtgaaaggagttgctaaagcaaaatggctgtttcaggagcaactgttgaggccggtggcagaggagggagcactgatgagggaggaggaagcactgatgcgggaggaggggcaggggaaggcgttgtgcaggttgaggtgggacaggggttgaagagcccggggaagagtctcctgaggtctaggaaggaatggacatgctcacctgaagggaccaggataggttggttaccagcacagtggcagtggctttggagccagagaccaacactagccatcaaggcaggcaaggatcaacaggctcctccagagaagggcctgtcaggaggaaaggcttggaagtgtgaaggaaggagctgcggccgactgagttgagttctgcccaagatctcactgcctgtgaaagagccagatggagactcaggtcagtcaggcctagtgcagcaatctctttcacgggagcacacagtaagcccaccactgctgtgcattgagaggagatcagggcacgctcagagccacagtgagcctttaggtctgggcaggaagcttccagcagttcactcgctgtgagttctacctgtgacacctcaccatgaccccaggtgtcccctgaggtatttctctggcatcttggagggagcagatcgccctagggagataaggagagaactggattattgacgactcactcctctacaaacatttaatgaacgcctactgaatggagccttgtgctgagtattaggggtatggtaatatgtctggaccccctttctgccctgatggggagagaggaacctttccaaacatccacacaaaacctattttagggatgtgaaaatgagtgacaattagcgatggggccaagctctctctttttgtcagtaacgaggtcattcatcactcattgggtgacacctccatccccagcactgcttcgggttgtggggggcaaatgcatccatgaacccaagactagaggtgggtgtggaacaaggtaatggtggctaggggagcccccattctggtgagttggtaaagtatgatgggggtgtttcaatcccacagcggctccccctgaaaatcagagcgaggttccagagctccgccttaggggttgggcttctggcagccaagtaccgccccttctaccccaagatgggatataaagtctcttagctccttccagagaagagtaagaagaaagaaaccatcacaggacctctaaggaagagaggaattgtacactcaccgaattc

SEQ ID NO: 4 - DNA, 18 bp, Artificial Sequence
  Primer
ggatcccctggagctgga

SEQ ID NO: 5 - DNA, 23 bp, Artificial Sequence
  Primer
gaattcggtgagtgtacaattcc